(19) United States
(12) Patent Application Publication
(10) Pub. No.: US 2026/0309482 A1
(43) Pub. Date: Oct. 8, 2026

(54) HOT END GLASS CONTAINER THICKNESS EVALUATION

(71) Applicant: Owens-Brockway Glass Container Inc., Perrysburg, OH (US)

(72) Inventors: Paul Arriz Tisoc, Lima (PE); Udaya Vempati, Perrysburg, OH (US); Tim Kohler, Monclova, OH (US); Peter Severson, Monclova, OH (US)

(21) Appl. No.: 19/173,805

(22) Filed: Apr. 8, 2025

(51) Int. Cl. G01B 11/06; G01J 5/00; G01J 5/52

(52) U.S. Cl. CPC G01B 11/0691; G01J 5/0022; G01J 5/52

(57) ABSTRACT

A method of evaluating a glass container in a hot end of a glass container manufacturing system involves generating a radiation intensity profile of the examined glass container and determining a thickness reading of the examined glass container based on the radiation intensity profile of the examined glass container and a radiation thickness correlation model that translates the radiation intensity profile of the examined glass container into the thickness reading. The radiation thickness correlation model is determined by comparing a radiation intensity profile of a sample glass container and one or more calibrating thickness measurements of the sample glass container and generating the radiation thickness correlation model based off an identified correlation between the radiation intensity profile and the calibrating thickness measurement(s) of the sample glass container. A hot end glass container evaluation system is also disclosed.

### TECHNICAL FIELD

This patent application discloses innovations related to evaluating a glass container in the hot end of a glass container manufacturing system and, more particularly, to examining the thickness of the glass container.

### BACKGROUND

Glass container manufacturing involves melting a batch feed material into molten glass and forming a container from a charge of the molten glass. The glass container includes a glass wall that provides the glass container with its overall shape and capacity. The glass wall must typically meet or exceed a specified minimum wall thickness so that the container can be reliably packaged, shipped, and filled. The uniformity of the wall thickness may be an indicator of the quality of the manufactured container and/or the level of control over the container forming process. Indeed, glass containers with a narrower glass wall thickness distribution can be formed from less glass, while still meeting the specified minimum wall thickness, compared to the same glass containers but with a wider glass wall thickness distribution. Advancements in process monitoring equipment, data collection, data analysis, and the general understanding of how glass flows while being shaped are allowing glass manufactures to exercise better control over the glass wall thickness uniformity of manufactured glass containers.

The glass container manufacturing operation is generally considered to have two phases: (i) the hot end and (ii) the cold end. The hot end is where the glass batch material, which includes raw materials and optionally cullet, is melted to produce molten glass and is also where glass containers are formed from charges of the molten glass. The glass batch material may be melted in a glass melting apparatus (e.g., a furnace or melter) and the resultant molten glass may be subsequently refined/fined to remove bubbles from the glass and then conditioned in a forehearth to thermally homogenize the glass and bring the glass to an appropriate viscosity for forming. Thereafter, the molten glass is cut into molten glass charges and the charges are distributed to a forming machine such as, for example, an individual section (IS) forming machine that includes a plurality of forming sections. Each forming section of an IS forming machine receives one molten glass charge, or a plurality of molten glass charges simultaneously, and forms each of the received charges into a glass container. After being formed, the still hot glass containers may be coated with a hot end coating (e.g., a tin oxide coating) and annealed in an annealing lehr. Annealing involves reheating the glass containers and then cooling the containers at a controlled rate to relieve internal stresses within the glass.

The cold end follows the hot end and begins after the glass containers emerge from the annealing lehr. At the cold end, the glass containers may be coated with a cold end coating (e.g., a polyethylene wax) and are inspected and packaged. Current cold end inspection equipment utilizes any of a variety of lasers, imagers, and/or other devices to verify that each glass container is free of commercially unacceptable defects and meets predetermined criteria for size, shape, thickness, and any other characteristics of interest. Glass containers that do not pass inspection are rejected and scrapped. Accordingly, in a traditional glass container manufacturing operation, the baseline form and quality of the glass containers are established in the hot end, as this is where the containers are initially formed from molten glass, while the finishing stages of the production process, including inspecting the physical characteristics and quality of the manufactured containers, are performed in the cold end.

Cold end inspection equipment is quite effective at identifying glass containers that do not meet the specified criteria for a commercially acceptable container and should therefore be rejected. This inspection equipment, however, is not well suited to provide real-time feedback about the state of the hot end operation since there is an appreciable time lag between the initial formation of a glass container in the hot end and the eventual inspection of that same glass container in the cold end. Indeed, by the time operations personnel discover through cold end inspection that one or more process conditions occurring in the hot end may be resulting in or at least contributing to the manufacture of non-conforming glass containers, a large number of glass containers are likely to have already been produced in the hot end under the deviated process condition(s). The cold end inspection lag means that a large quantity of manufactured glass containers sometimes have to be scrapped once a recurring anomaly in the manufactured glass containers is identified during cold end inspection. The ability to inspect glass containers in the hot end would allow for the earlier identification of trends and/or potential issues that my lead to glass containers failing inspection. The obtained inspection information may be used to adjust one or more process conditions in the hot end to avoid producing glass containers that fail inspection in the first place or, at the very least, to limit the quantity of manufactured containers that need to be scrapped after an issue is identified.

Accurately measuring the physical attributes of glass containers in the hot end is complicated due to the high temperatures of the glass containers and the dynamic nature of the container manufacturing process. In the hot end, the glass containers are too hot for manual handling and are generally still in a semi-deformable state. As such, the deployment of conventional thickness measurement techniques is customarily relegated to the cold end. For example, the use of conventional offline thickness measurement tools in the hot end would require glass containers to be collected and inspected through an analytical approach that is too slow to keep up with container production speeds and, furthermore, would require the collected containers to be annealed prior to being measured. Other non-contact methods, such as laser scanning, may not be reliable and/or precise enough for proper measurements, especially when affected by the thermal radiation emitted by the hot glass. An improved method and system that provides prompt, accurate, non-contact thickness readings of glass containers in the hot end in real-time is therefore needed. The ability to attain such real-time hot end glass thickness readings may allow variances in the manufacturing process to be identified and addressed in a more timely manner as opposed to reacting to the discovery of potential issues during cold end inspection.

### SUMMARY

A hot end glass container evaluation system and a method of evaluating one or more glass containers in a hot end of a glass container manufacturing system are disclosed. The hot end glass container evaluation system includes at least two infrared image sensors and a processing system. Each of the infrared image sensors is configured to capture an infrared image of an examined glass container in the hot end of the glass container manufacturing system and the processing system is configured to received the captured infrared images from the infrared image sensors. The processing system generates a radiation intensity profile of the examined glass container and determines a thickness reading of the examined glass container based on the radiation intensity profile and a radiation thickness correlation model that translates the radiation intensity profile into the thickness reading. A method of evaluating a glass container in the hot end of a glass container manufacturing system includes generating a radiation intensity profile of an examined glass container and determining a thickness reading of the examined glass container based on the radiation intensity profile of the examined glass container and a radiation thickness correlation model, which may be determined based on the one or more calibrating thickness measurements of a sample glass container and a radiation intensity profile of the sample glass container.

### BRIEF DESCRIPTION OF THE DRAWINGS

FIG. 1 is a diagrammatic illustration of a hot end glass container evaluation system incorporated into a hot end of a glass container manufacturing system according to an illustrative embodiment of the present disclosure.

FIG. 2 is a diagrammatic illustration of the hot end glass container evaluation system additionally showing an offline thickness measurement tool that is part of the system.

FIG. 3 is a flowchart depicting a radiation thickness correlation determination process for determining the radiation thickness correlation model according to an illustrative embodiment of the present disclosure.

FIG. 4 is a schematic, diagrammatic flowchart depicting an exemplary radiation thickness correlation determination process using an infrared image from each of a plurality of infrared image sensors of the hot end glass container evaluation system depicted in FIG. 1.

FIG. 5 depicts left and right groups of infrared images, with the left column corresponding to a first group of infrared images for a first infrared image sensor and the right column corresponding to a second group of infrared images for a second infrared image sensor, and each group of infrared images having three images depicting the same glass container with the top image being the captured infrared image, the middle image being a smoothed image of the original image, and the bottom image being a sparse component of the captured infrared image.

FIG. 6 depicts a schematic, sectional, top plan view of the two infrared image sensors of the hot end glass container evaluation system depicted in FIG. 1, with each sensor facing and capturing an infrared image of a cylindrical body portion of a glass container and being offset from one another by an offset angle, and additionally depicting the corresponding infrared image of each image sensor in relation to the cylindrical body portion of the glass container.

FIG. 7 depicts an exemplary graphical representation of a radiation intensity profile of a glass container according to one embodiment of the present disclosure.

FIG. 8 is a flowchart illustrating a thickness reading determining process for determining a thickness reading of a glass container in a hot end of a glass container manufacturing system according to one embodiment of the present disclosure.

FIG. 9 is a side perspective view of a generalized glass container that is representative of the glass containers formed in the hot end of the glass container manufacturing system.

### DETAILED DESCRIPTION

The present disclosure describes a hot end glass container evaluation system and a related method for evaluating the thickness of glass containers (e.g., bottles and jars) in real-time at the hot end of a glass container manufacturing system. The hot end glass container evaluation system remotely determines the intensity of infrared radiation emitted from a glass container, preferably using at least two infrared image sensors, and generates a thickness reading of the glass container from that information. The thickness reading is a thickness profile representative of the predicted thickness of the glass around the container and is inferred from the intensity of infrared radiation emitted from the glass container. The hot end glass container evaluation system leverages an offline measurement tool that provides one or more calibrating thickness measurements of at least one sample glass container, which are used to generate a radiation thickness correlation model that is representative of an identified correlation between the calibrating thickness measurement(s) and infrared radiation emitted from the sample container.

The hot end glass container evaluation system can remotely determine a thickness reading for each glass container being examined by capturing infrared images of the glass container while in the hot end, without having to physically disturb the glass container, and then processing those images. In other words, the glass container does not have to be removed, for example, from a forming conveyor that is transporting the glass container from the IS forming machine to the annealing lehr. The infrared images of the glass container being examined are used to generate a radiation intensity profile of the container. The thickness reading of the glass container is then computed from the radiation intensity profile and the radiation thickness correlation model. The radiation thickness correlation model quantifies a correlation between radiation intensity and glass thickness and is used to convert the radiation intensity profile of the glass container into the thickness reading for the container. The radiation thickness correlation model is established and periodically updated or calibrated by associating the radiation intensity profile of one or more representative sample glass containers with one or more offline thickness measurements, referred to here as one or more calibrating thickness measurements, of those container(s). The hot end glass container evaluation system can be operated to determine the thickness reading for each and every glass container that is produced or some predetermined set or sequence of containers, such as every other container, every third container, or a batch of some number of consecutive containers every so often, etc. The thickness reading can be analyzed to detect anomalies such as thin or thick regions of the glass wall, defects, or other notable physical features in real time, thus allowing corrective measures to be implemented to address issues in the manufacturing process that may be causing or at least contributing to the observed inconsistencies.

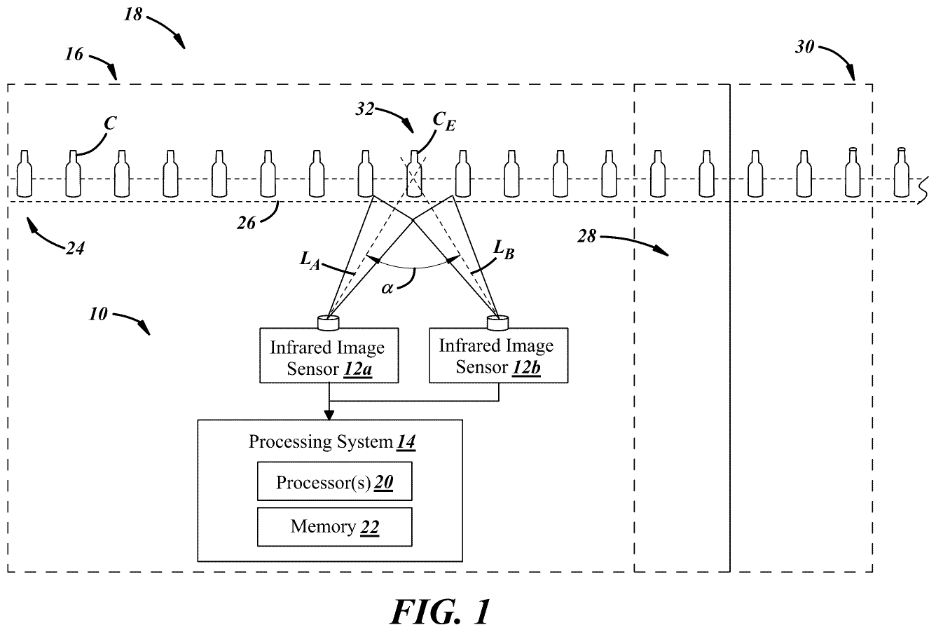

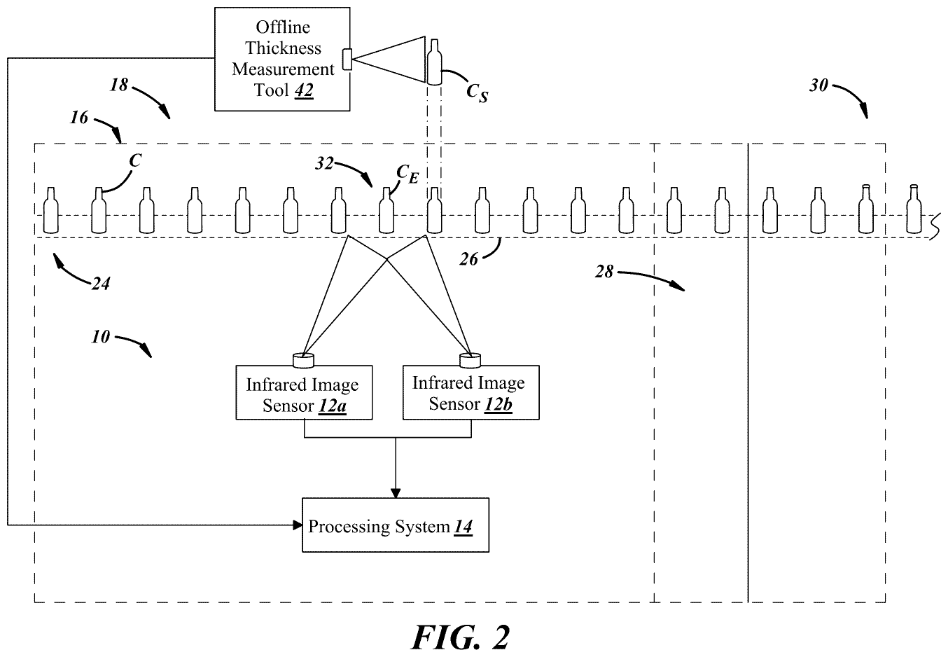

FIGS. 1-2 depict a hot end glass container evaluation system 10, which includes at least two infrared image sensors 12a, 12b and a processing system 14. Each of the infrared image sensors 12a, 12b is configured to capture at least one infrared image of a glass container C_{E} being examined (sometimes referred to as an “examined glass container”) by the system 10 as the glass container C_{E} passes through a hot end 16 of a glass container manufacturing system 18. The processing system 14 includes at least one processor 20 and memory 22 storing computer instructions that, when executed by the at least one processor 20, cause the processing system 14 to (i) determine the radiation intensity profile of the examined glass container C_{E} based on the infrared images of the glass container C_{E} and (ii) determine the thickness reading of the examined glass container C_{E} based on the radiation intensity profile and the radiation thickness correlation model. The hot end glass container evaluation system 10 may be implemented into the manufacturing system 18 if, for example, real-time visibility into the distribution of glass within glass containers C manufactured by the glass container manufacturing system 18 is desired shortly after the containers C are formed in the hot end 16. The hot end glass container evaluation system 10 may be integrated with both existing and newly developed glass manufacturing lines, providing real-time, actionable data that can enhance quality control, reduce material waste, and improve the overall efficiency of the glass container manufacturing process.

The hot end 16 of the glass container manufacturing system 18 is where the glass containers C are initially formed from molten glass. The hot end 16 includes a glass melting apparatus that receives and melts a vitrifiable batch feed material into molten glass. The glass melting apparatus may be a continuous melting furnace, a submerged combustion melter, or some other apparatus that produces molten glass. In a continuous melting furnace, the vitrifiable batch feed material is fed onto a molten glass bath within an upstream melting chamber of the furnace and heat is provided to the melting chamber above the glass bath in an open combustion zone by overhead burners. In contrast, in a submerged combustion melter, the vitrifiable batch feed material is fed into a glass melt that is agitated and heated by combustion products that are fired directly into the glass melt by submerged burners. The produced molten glass is then refined or fined, either in a refining chamber of a continuous melting furnace or in a separate finer positioned downstream of a submerged combustion melter, and delivered to a forehearth where the glass is thermally conditioned for subsequent glass forming operations. The forehearth provides the molten glass to a glass feeder, which modulates molten glass through an exit orifice of the feeder. The molten glass exiting the orifice is cut by shears or some other cutting device to produce a discrete charge or “gob” of molten glass.

The gobs of molten glass are distributed to a forming area 24 that includes one or more glass forming machines such as one or more individual section (IS) machines. Each IS machine includes one or more forming sections. In each forming section, a blank side includes one or more blank molds and a blow side includes one or more blow molds. Each of the blank mold(s) on the blank side receives a gob of molten glass and forms the glass gob into a parison (a partially formed container), and each of the blow molds on the blow side receives a parison from the blank side and forms the parison into a glass container C, as is generally well understood in the art. After being formed at the forming area 24, the hot glass containers C are removed from their respective forming machines and placed on a forming conveyor 26. The forming conveyor 26 conveys the glass containers C away from the forming area 24 and to an annealing lehr 28 where the glass containers C are reheated and then cooled at a controlled rate to relieve internal stresses within the glass. The glass containers C may also be coated with a hot end coating prior to or while passing through the annealing lehr 28.

A cold end 30 of the glass container manufacturing system 18 follows the annealing lehr 28 of the hot end 16. In the cold end 30, the glass containers C may be coated with a cold end coating, if desired, and are inspected and packaged. The inspection in the cold end 30 employs sophisticated inspection equipment that analyzes the glass containers C to ensure the containers C satisfy certain predetermined criteria or specifications set by the manufacturer and/or customer, or some other interested entity, and to manage an internal quality control program. For example, in the cold end 30, the glass containers C are typically inspected for the correct size, shape, glass wall thickness, and to verify that commercially unacceptable defects (stones, blisters, or other visual irregularities) are not present. Any glass containers C that fail the cold end inspection process are typically rejected and scrapped. As previously explained, however, the inspection equipment in the cold end 30 is unable to provide real-time feedback on the quality of the glass containers C as the containers are produced in hot end 16, particularly with respect to glass wall thickness, due to the lag between the initial formation of the glass containers C in the hot end 16 and the eventual inspection of the same glass containers C in the cold end 30. This lag between container formation and container inspection can vary but, in many instances, ranges anywhere from 20 minutes to 60 minutes. In that regard, cold end inspection equipment usually cannot help operations personnel monitor and spot trends, changes, and/or expectations in how glass is distributed within the manufactured glass containers C quick enough so that corrective adjustments can be made in the hot end 16 before a large number of the glass containers C have to be scrapped for failing inspection in the cold end 30.

In the disclosed glass container manufacturing system 18, an evaluation zone 32 is provided after the forming area 24 but before the annealing lehr 28. The forming conveyor 26 that transports the glass containers C to the annealing lehr 28 conveys some or all of the glass containers C through the evaluation zone 32 so that at least some of the recently-formed glass containers C can be evaluated by the glass container evaluation system 10 in the hot end 16. Those glass containers C that are evaluated by the glass container evaluation system 10 are referred to as the examined glass containers C_{E}. Accordingly, without necessarily having to disturb the production output of the manufacturing system 18, the infrared images of the glass containers C_{E} being examined are captured by the infrared image sensors 12a, 12b while the glass containers C_{E} are on the forming conveyor 26. The processing system 14 extracts infrared radiation intensity data from the captured infrared images of each examined container C_{E} and determines the thickness reading of a glass wall W (FIG. 9) of the container C_{E}.

The thickness reading provides an accurate prediction of the thickness of the glass wall W of the examined glass container C_{E} at one or more locations of the glass wall W and, preferably, at a plurality of locations of the glass wall W that are spaced apart around a central longitudinal axis X (FIG. 9) of the container C_{E} so that a glass wall thickness distribution profile of the container C_{E} can be discerned. In this sense, the glass container evaluation system 10 may be considered an “online” hot end system because it evaluates the examined glass containers C_{E} in the hot end 16 and provides real-time glass distribution/thickness data that can help operations personnel (or an operations program) better control the production of glass containers C in the hot end 16. The thickness reading can be analyzed to detect anomalies such as thin or thick regions of the glass wall W, defects, or other notable physical features in real time, thus allowing corrective measures to be implemented to address issues in the manufacturing process that may be causing or at least contributing to the observed inconsistencies. Moreover, if the thickness reading is deemed accurate enough, the hot end glass container evaluation system 10 may be operated as an inspection system in which accept/reject decisions are made for each glass container C, possibly allowing the cold end inspection equipment to be eliminated.

Each infrared image sensor 12a, 12b is a sensor that captures emitted infrared (IR) radiation. The captured emitted IR radiation may be represented as an array of pixels that together constitute an infrared image, which is an example of a single-channel or grayscale image. As used herein, “infrared” or “IR” includes near-infrared (near IR) radiation, mid-infrared (mid-IR) radiation, and far-infrared (far IR) radiation, which spans electromagnetic radiation having wavelengths from 750 nm to 1 mm. Each infrared image sensor 12a, 12b may, as shown, be a passive IR (PIR) sensor configured to receive infrared light emitted from the examined glass container C_{E}, which is then converted from its analog representation to a digital representation. In other embodiments, one or both of the infrared image sensors 12a, 12b may be an active IR sensor. Each infrared image sensor 12a, 12b, moreover, may be part of a conveying system, possibly implemented as a line scanner or as a 2D sensor capable of capturing an entire image at once. Each infrared image sensor 12a, 12b may be or include a charge-coupled device (CCD) sensor, a complementary metal-oxide semiconductor (CMOS) sensor, or any other suitable type of sensor capable of capturing infrared radiation in image form. Various processing techniques may be used to prepare the infrared image for downstream processing, including, for example, reducing noise or enhancing contrast.

The infrared images captured by the infrared image sensors 12a, 12b are processed by the processing system 14 and, more specifically, the processor 20, to generate a radiation intensity profile of the examined glass container C_{E}. Considering the infrared radiation emitted from any location of the glass wall W is a function of the amount of glass and the temperature of the glass at that location, the radiation intensity profile depicts variations in emitted infrared radiation from around the glass wall W of the examined glass container C_{E} within a specified target segment T (FIG. 9) and corresponds to an intensity map of pixel values representative of the infrared radiation sensed by the infrared image sensors 12a, 12b. The target segment T may, for example, be an annular band that surrounds the central longitudinal axis X of the examined glass container C_{E} and ranges from 10 mm to 150 mm, or more narrowly from 70 mm to 130 mm, in axial height along the central longitudinal axis X of the glass container C_{E}. The target segment T may be located anywhere within a body portion 34 of the glass wall W of the examined glass container C_{E} that extends between a base 36 of the glass wall W and a shoulder 38 of the glass wall W as shown, for example, in FIG. 9. The body portion 34 of the glass wall W surrounds and extends along the central longitudinal axis X of the examined glass container C_{E} and may be cylindrical with a circular cross-section when sectioned perpendicular to the axis X or it may assume some other circumferentially closed shape.

The radiation intensity profile of the examined glass container C_{E} may be generated in a way that mitigates non-linear interference resulting from the glass container C_{E} having a front side and a superposed back side when imaged from a side profile of the container C_{E}. For example, an interior space of the examined glass container C_{E} is defined by the glass wall W, which is circumferentially closed in that the glass wall W encircles or otherwise envelopes the interior space on all sides except for an opening at the mouth 40 (FIG. 9) of the container C_{E}, as is true for all of the glass containers C manufactured in the hot end 16. When viewing a side profile of the examined glass container C_{E}, especially if the glass wall W is transparent, both the front portion of the glass wall W that is closest to the viewer and the back portion of the glass wall W that is farthest from the viewer and behind the front portion are visible. The non-linear radiation interference caused by the superposition of the front and back portions of the examined glass container C_{E} may be resolved during container evaluation by capturing and processing at least two different angularly displaced infrared images of the glass container C_{E}; that is, a first infrared image presents a first portion of the examined glass container C_{E} and a second infrared image presents a second portion of the container C_{E} that is angularly displaced from the first portion about the central longitudinal axis X of the container C_{E}. In other words, two infrared images of the examined glass container C_{E} are “angularly displaced” when different portions of the glass container C_{E} are shown in the images such that the container C_{E} appears to have been rotated about its central longitudinal axis X when comparing the two images.

The multiple angularly displaced infrared images of the examined glass container C_{E} may be captured by the two infrared image sensors 12a, 12b. Indeed, during evaluation of each glass container C_{E} being examined, the processing system 14 receives and processes at least one infrared image from each of the infrared image sensors 12a, 12b, both of which captures their respective image(s) as the glass container C_{E} is being conveyed through the evaluation zone 32. The two infrared image sensors 12a, 12b are spaced apart and may be angularly offset relative to a reference point that coincides with the examined glass container C_{E} being imaged. The angular spacing of the infrared image sensors 12a, 12b is represented by an offset angle α, which is the angle between a first sightline L_{A} of the first infrared image sensor 12a and a second sightline L_{B} of the second infrared image sensor 12b. The first and second sightlines L_{A}, L_{B} extend from a center of the first infrared image sensor 12a and a center of the second infrared image sensor 12b, respectively, to the examined glass container C_{E} (i.e., the reference point). The offset angle α is measured in a plane that intersects the first and second sightlines L_{A}, L_{B}. This plane may be horizontal and thus be defined in the X-Y (ground) plane or it may be angled with respect to the horizontal. The offset angle α between the two infrared image sensors 12a, 12b may be 10 degrees or greater, 30 degrees or greater, 60 degrees or greater, 90 degrees or greater, or even 180 degrees or greater. When imaging the examined glass container C_{E}, the first and second infrared image sensors 12a, 12b are preferably configured to simultaneously capture their respective infrared images of each glass container C_{E} being examined while the glass container C_{E} is at a particular location within the inspection zone 32.

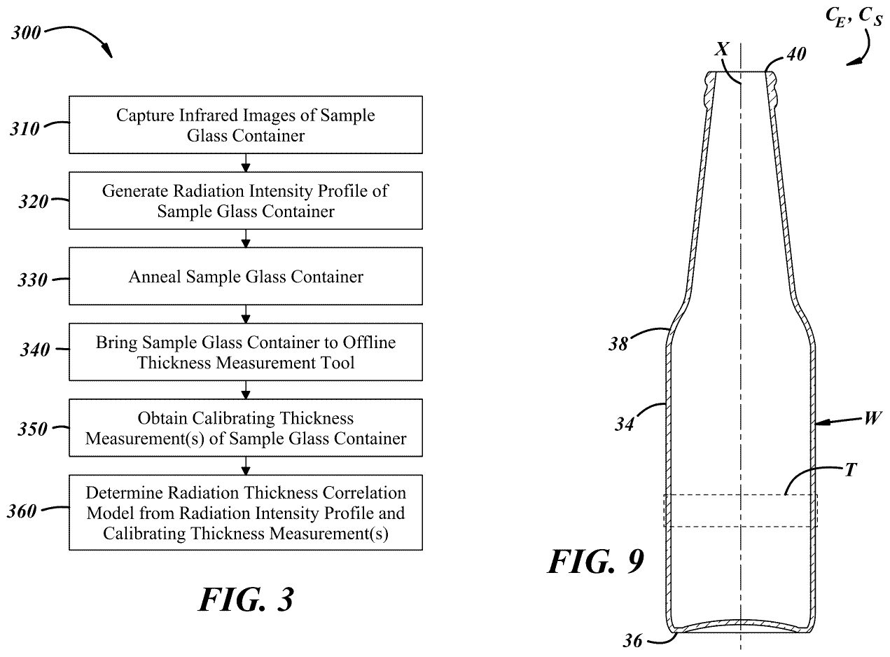

The processing system 14 receives the infrared image(s) captured by each of the infrared image sensors 12a, 12b and is configured to determine the thickness measurement of the examined glass container C_{E} from those infrared images. The processing system 14 extracts infrared image data from the infrared images within the target segment T of the examined glass container C_{E}, processes the infrared image data, and generates the radiation intensity profile of the examined glass container C_{E} from the infrared image data. The processing system 14 then determines the thickness reading of the examined glass container C_{E} from the radiation intensity profile of the container C_{E} and the current radiation thickness correlation model—the radiation thickness correlation model is preferably routinely updated to ensure that the thickness reading computed from the radiation intensity profile accurately coincides with offline calibrating thickness measurement(s). The radiation thickness correlation model is determined using a radiation thickness correlation determination process 300 (FIG. 3), and the thickness measurement of the glass container is determined using a thickness reading determination process 800 (FIG. 8), each of which is discussed in more detail below.

The hot end glass container evaluation system 10 further includes an offline thickness measurement tool 42 as is shown in FIG. 2. The offline thickness measurement tool 42 is used to acquire the calibrating thickness measurement(s) of at least one sample glass container C_{S} that is removed from the forming conveyor 26 and analyzed by the tool 42. The one or more calibrating thickness measurements of the sample glass container C_{S} includes at least one calibrating thickness measurement of the sample glass container C_{S} within the same target segment T used to generate the radiation intensity profile and, preferably, is a set of calibrating thickness measurements taken from a plurality of locations disposed around the central longitudinal axis X of the container C_{S} within the target segment T. Each calibrating thickness measurement is a quantified numerical value representing the thickness of the glass wall W at a given location and the chosen location(s) of the calibrating thickness measurement(s) may be random or part of a predetermined distribution of measurement locations. The calibrating thickness measurement(s) are used to establish the radiation thickness correlation model, which the processing system 14 references to determine the thickness reading of the glass containers C_{E} being examined, by comparing the radiation intensity profile of the sample glass container C_{S} and the calibrating thickness measurement(s) of the same container C_{S} obtained from the offline thickness measurement tool 42 and ascertaining the correlation between the radiation intensity profile and the calibrating thickness measurement(s).

The offline thickness measurement tool 42 is any device that can measure the thickness of the glass wall of the one or more sample glass containers C_{S}. In one embodiment, the offline thickness measurement tool 42 employs a computed tomography scanner that can generate a manipulable (e.g., rotatable and dissectible) 3-dimensional digital model of the sample glass container C_{S} with a fully mapped glass thickness from a plurality of X-rays of the container C_{S}. The digital model of the sample glass container C_{S} allows for the glass wall thickness of the sample glass container C_{S} to be measured at any of a variety of desired locations to a high degree of precision. One particular offline thickness measurement tool that employs a computed tomography scanner is described in U.S. Pat. No. 10,890,477, which is hereby incorporated by reference in its entirety and is attributed to the offline thickness measurement tool 42 as an exemplary embodiment of the tool 42. Such a tool is commercially available from Tiama (www.tiama.com) under the product name XLAB. The offline thickness measurement tool 42 may be located in the hot end 16 or the cold end 30 of the glass container manufacturing system 18. And since the hot end glass container evaluation system 10 seeks to correlate infrared radiation intensity with glass thickness of a handleable glass container, the sample glass container C_{S} may be imaged in the offline thickness measurement tool 42 and the digital model of the sample glass container C_{S}, which is used to obtain the calibrating thickness measurement(s), may be constructed with the sample glass container C_{S} being at any temperature that is appropriate for the tool 42.

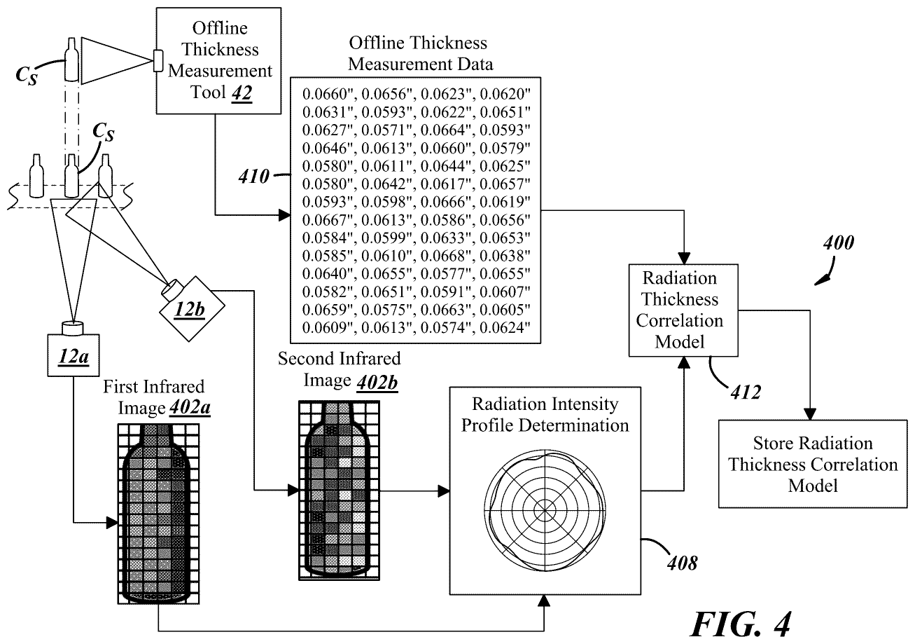

The radiation thickness correlation model—both during the initial establishment of the correlation model and during a calibration routine in which the correlation model is updated—is determined by a radiation thickness correlation determination process 300 as shown, for example, in FIGS. 3-4. In this process, which is represented generally in FIG. 3 and identified by reference numeral 300, infrared images of the at least one sample glass container C_{S} are captured by the infrared image sensors 12a, 12b (step 310) and the radiation intensity profile of the sample glass container C_{S} is generated by the processing system 14 from the infrared images (step 320). After being imaged in the inspection zone 32, the sample glass container C_{S} is annealed (step 330). The sample glass container C_{S} is brought to the offline measurement tool 42 (step 340) after annealing and, through the measurement procedure employed by the tool 42, the calibrating thickness measurement(s) of the sample glass container C_{S} are obtained (step 350). The calibrating thickness measurement(s) of the sample glass container C_{S} are received by processing system 14, which, in turn, determines the radiation thickness correlation model based off a comparison of the radiation intensity profile and the calibrating thickness measurement(s) of the sample glass container C_{S} (step 360). The processing system 14 calculates the radiation thickness correlation model based on an identified correlation between radiation intensity and measured glass wall thickness, which may be a learned correlation, so that the radiation intensity profiles of the glass containers C_{E} being examined can be translated or converted into accurate real-time thickness readings. This process 300 may be repeated more than once to help ensure continued refinement of the radiation thickness correlation model over time.

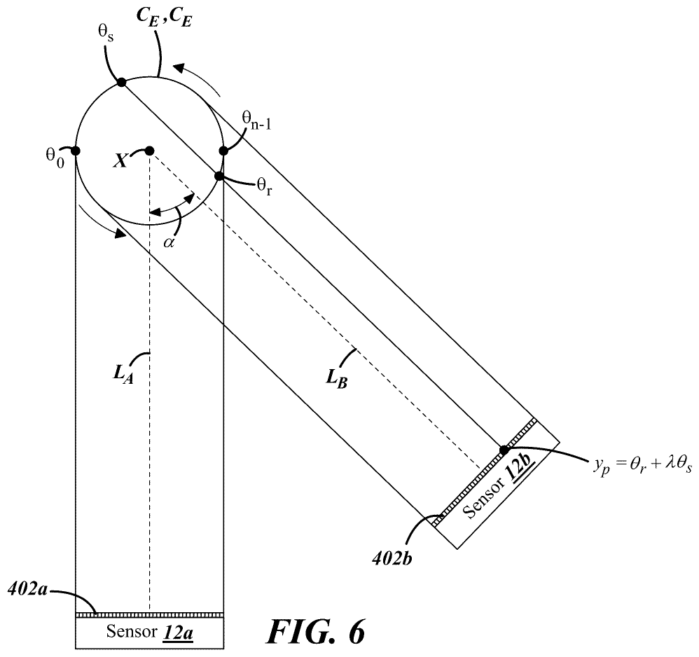

As a more specific implementation of the radiation thickness correlation determination process 300, a flowchart is provided in FIG. 4 that depicts a process flow 400 that may be used to perform certain steps of the radiation thickness correlation determination process 300. To perform step 310, for instance, the first infrared image sensor 12a captures a first infrared image 402a of the sample glass container C_{S} and the second infrared image sensor 12b captures a second infrared image 402b of the sample glass container C_{S}. The captured infrared images 402a, 402b from the first and second infrared image sensors 12a, 12b are of different angularly displaced portions of the sample glass container C_{S}. For example, with reference now to FIG. 6, the first infrared image sensor 12a used to capture the first infrared image 402a and the second infrared image sensor 12b used to capture the second infrared image 402b may be disposed at different first and second image sensing positions, respectively. Preferably, as shown, the first and second infrared image sensors 12a, 12b may be angularly offset by an offset angle α (also referred to as multi-sensor field of view angle) measured between their respective sightlines L_{A}, L_{B}, which intersect at the central longitudinal axis X of the sample glass container C_{S}, of about (±5°) 45 degrees. Indeed, when the sample glass container C_{S} has a circular cross-section as depicted in FIG. 6, the captured first and second infrared images 402a, 402b of the container C_{S} may be circumferentially offset about the central longitudinal axis X of the sample glass container C_{S}.

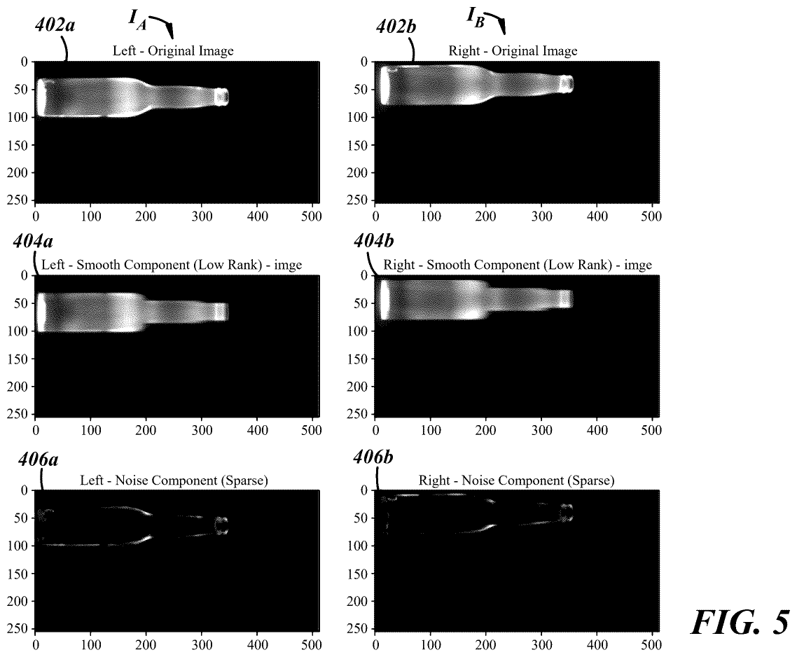

Next, to generate the radiation intensity profile 408 (FIG. 4) of the sample glass container C_{S} (step 320), the first and second captured infrared images 402a, 402b are delivered to, and received by, the processing system 14 from the first and second infrared image sensors 12a, 12b, respectively. The first and second infrared images 402a, 402b captured by the first and second infrared image sensors 12a, 12b are shown for a particular sample glass container C_{S} in FIG. 5. In FIG. 5, two groups of infrared images I_{A}, I_{B} are shown. The “Left” images are a first group of infrared images I_{A} associated with the first infrared image sensor 12a and the “Right” images are a second group of infrared images I_{B} associated with the second infrared image sensor 12b. The first and second infrared images 402a, 402b captured by the first and second infrared image sensors 12a, 12b are shown at the top of the first and second groups of infrared images I_{A}, I_{B}. The radiation intensities expressed in each of the captured infrared images 402a, 402b of the sample glass container C_{S} contains the superposition of the back side of the container C_{S} (the portion of the container behind the front side) and the front side of the container C_{S} (the portion of the container directly in front of the infrared image sensor) and may additionally include a sparse component. Radiation intensity attributable to the sparse component may optionally be removed from the first and second captured infrared images 402a, 402b by the processing system 14 prior to generating the radiation intensity profile 408.

To remove the sparse component, the processing system 14, after receiving the captured infrared images 402a, 402b from the infrared image sensors 12a, 12b, processes each of the first and second captured infrared images 402a, 402b to derive a first smoothed infrared image 404a and a second smoothed infrared image 404b, respectively. The smoothed infrared images 404a, 404b are derived by removing radiation intensity attributable to sparse surface features of the glass wall W of the sample glass container C_{S}. Sparse surface features are non-smooth surface features that interrupt an otherwise smooth surface of the glass wall W and may include sparse perturbations in the borders/peripherals/edges of the container C_{S}, protrusions and/or intrusions due to embossments, etchings, wells, or cavities, container identifiers (CIDs) such as a dot matrix code and/or other manufacturer details, and/or decorations. For example, in FIG. 5, a sparse component 406a of the first captured infrared image 402a (bottom-left of FIG. 5) and a sparse component 406b for the second captured infrared image 402b (bottom-right of FIG. 5) are shown. These sparse components 406a, 406b are removed from the first and second captured infrared images 402a, 402b by the processing system 14 to yield, respectively, the first and second smoothed infrared images 404a, 404b, which are shown in the middle row of the groups of images I_{A}, I_{B}.

The radiation intensity observed in the captured infrared images 402a, 402b or, if removal of the sparse component is desired, the smoothed infrared images 404a, 404b, is reconciled within at least the specified target segment T of the container C_{S} to generate the radiation intensity profile 408 of the container C_{S}. The radiation intensities of the infrared images 402a, 402b (or 404a, 404b), which may be represented within the target segment T by a two-dimensional array of pixel values, are reconciled by solving for the superposition of the of the front and back portions of the glass wall W and allocating radiation intensity values fully around the glass wall W. The superposition of the glass wall W can be modeled for each pixel as additive with an unknown attenuation factor for either the front or back side of the glass wall W such that a given radiation intensity value for each pixel corresponds to the superposition of two unknowns (back and front of the image). In the discussion that follows, and with reference to FIGS. 4 and 6, an approach for solving for the superposition of the of the front and back portions of the glass wall W and generating the radiation intensity profile 408 of the sample glass container C_{S} based on the first and second captured infrared images 402a, 402b is described, although, as explained above, the first and second smoothed infrared images 404a, 404b could just as easily be used in place of the captured infrared images 402a, 402b.

Referring back to FIG. 6, for example, the first and second captured infrared images 402a, 402b received from the first and second infrared image sensors 12a, 12b are shown projected onto their respective imaged portions of the sample glass container C_{S}. As indicated, a reference position of the sample glass container C_{S} is defined as θ₀ and corresponds to the first pixel on the left of the first captured infrared image 402a, which has n number of pixels in a horizontal row of pixels, and, therefore, the position half-way around the container C_{S} that corresponds to the last pixel on the right of the image 402a is defined as θₙ₋₁. As such, each horizontal row of n pixels in the first captured infrared image 402a contains radiation data for 2n positions around and along the full glass wall W including the superposed side—specifically, an intensity value for each of positions θ₀→θₙ₋₁ representing the front side of the glass wall W and an intensity value for each of positions θₙ₋₁→θ₂ₙ₋₁ representing the superposed back side of the glass wall W. The same positional references set for the first captured infrared image 402a are used for the second captured infrared image 402b so that data from the two images 402a, 402b can be reconciled. For each of the captured infrared images 402a, 402b, the radiation intensity (y) of each pixel (e.g., the intensity (yₚ) of pixel p in image 402b) is modeled as an additive of radiation intensity from a position on the front side (e.g., position θᵣ in image 402b) and a superposed position on the back side (e.g., position θₛ in image 402b) of the glass wall W with an attenuation factor (λ) representing the fraction of intensity from the back side that passes through the front side and is sensed by the infrared sensor 12a, 12b.

By reconciling the radiation intensity observed in captured infrared images 402a, 402b, all of the 2n radiation intensity values around the sample glass container C_{S} can be calculated and the radiation intensity profile 408 of the container C_{S} within the target segment T can be generated. Regarding the first captured infrared image 402a of the first infrared image sensor 12a, the radiation intensity (y₀) of the first pixel at the reference position (θ₀), which contains the radiation intensity from superposed positions θ₀ and θ₂ₙ₋₁, is modeled as:

$${y}_{0} = [ \begin{matrix}1 & 0 & 0 & … & 0 & 0 & λ\end{matrix} ] [ \begin{matrix}{θ}_{0} \\ {θ}_{1} \\ ⋮ \\ {θ}_{2 n - 1}\end{matrix} ]$$

This model can be extended across the full row of n pixels of the first captured infrared image 402a to allocate the radiation intensity in each pixel to its respective superposed positions of the glass wall W of the sample glass container C_{S}, as shown in the matrix (A₁) below:

$$[ \begin{matrix}{y}_{0} \\ {y}_{1} \\ {y}_{2} \\ ⋮ \\ {y}_{n - 2} \\ {y}_{n - 1}\end{matrix} ] = {[ \begin{matrix}1 & 0 & 0 & 0 & & 0 & 0 & 0 & 0 & 0 & 0 & & 0 & 0 & 0 & λ \\ 0 & 1 & 0 & 0 & … & 0 & 0 & 0 & 0 & 0 & 0 & … & 0 & 0 & λ & 0 \\ 0 & 0 & 1 & 0 & & 0 & 0 & 0 & 0 & 0 & 0 & & 0 & λ & 0 & 0 \\ & ⋮ & & & ⋱ & & & ⋮ & & & & ⋱ & & ⋮ & & \\ 0 & 0 & 0 & 0 & … & 0 & 1 & 0 & 0 & λ & 0 & … & 0 & 0 & 0 & 0 \\ 0 & 0 & 0 & 0 & & 0 & 0 & 1 & λ & 0 & 0 & & 0 & 0 & 0 & 0\end{matrix} ] [ \begin{matrix}{θ}_{0} \\ {θ}_{1} \\ {θ}_{2} \\ ⋮ \\ {θ}_{2 n - 2} \\ {θ}_{2 n - 1}\end{matrix} ]}_{2 n \times 1} \qquad Equation ( 1 )$$

$${Y}_{1} = {A}_{1} Θ$$

Similarly, for the second captured infrared image 402b, the radiation intensity of each pixel in the full row of pixels is allocated to its respected superposed positions of the glass wall W of the sample glass container C_{S}, as shown in the matrix (A₂) below in which the offset angle between the first and second infrared image sensors 12a, 12b is incorporated into matrix as a roll in one or more columns of the previous A₁ matrix:

$$[ \begin{matrix}{y}_{0} \\ {y}_{1} \\ {y}_{2} \\ ⋮ \\ {y}_{n - 2} \\ {y}_{n - 1}\end{matrix} ] = {[ \begin{matrix}0 & λ & 1 & 0 & & 0 & 0 & 0 & 0 & 0 & 0 & & 0 & 0 & 0 & 0 \\ λ & 0 & 0 & 1 & … & 0 & 0 & 0 & 0 & 0 & 0 & … & 0 & 0 & 0 & 0 \\ 0 & 0 & 0 & 0 & & 0 & 0 & 0 & 0 & 0 & 0 & & 0 & 0 & 0 & λ \\ & ⋮ & & & ⋱ & & & ⋮ & & & & ⋱ & & ⋮ & & \\ 0 & 0 & 0 & 0 & … & 0 & 0 & 0 & 1 & 0 & 0 & … & 0 & 0 & 0 & 0 \\ 0 & 0 & 0 & 0 & & 0 & 0 & 0 & 0 & 1 & λ & & 0 & 0 & 0 & 0\end{matrix} ] [ \begin{matrix}{θ}_{0} \\ {θ}_{1} \\ {θ}_{2} \\ ⋮ \\ {θ}_{2 n - 2} \\ {θ}_{2 n - 1}\end{matrix} ]}_{2 n \times 1} \qquad Equation ( 2 )$$

$${Y}_{1} = {A}_{1} Θ$$

Accordingly, for corresponding rows of pixels of the first and second captured infrared images 402a, 402b, the radiation intensity observed in the images 402a, 402b can be modeled for all of the 2n positions of the glass wall W of the sample glass container C_{S} as follows:

$$[ \begin{matrix}{Y}_{1} \\ {Y}_{2}\end{matrix} ] = [ \begin{matrix}{A}_{1} \\ {A}_{2}\end{matrix} ] Θ \qquad Equation ( 3 )$$

And, by joining the data models, the following linear system is obtained from which the radiation intensity at each of the 2n positions around the sample glass container C_{S} can be calculated within the particular corresponding rows of pixels:

$$Y = A Θ \qquad Equation ( 4 )$$

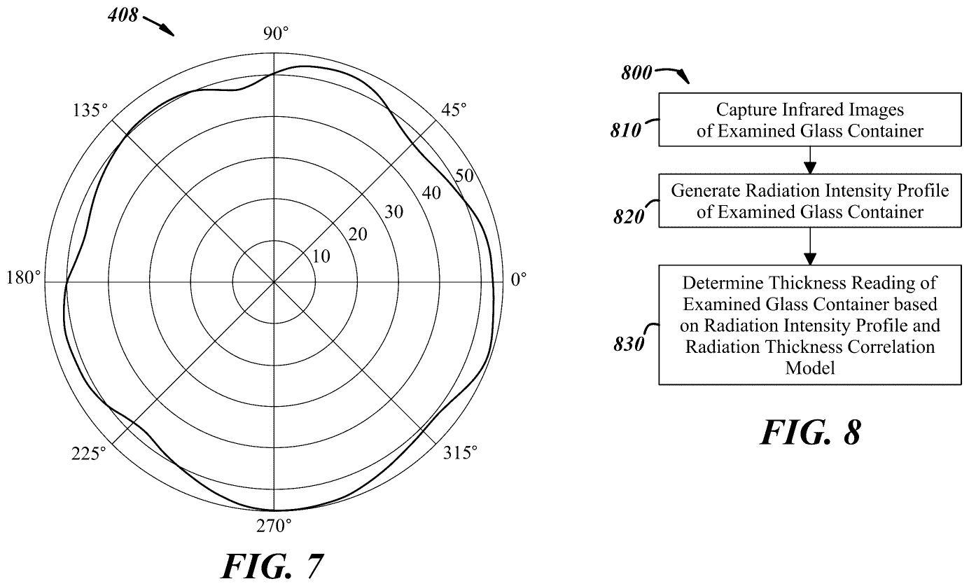

The image reconciliation procedure described above in connection with FIG. 6 may be applied to each horizontal row of pixels in the captured infrared images 402a, 402b within the target segment T and, from there, the radiation intensity profile 408 of the glass wall W of the sample glass container C_{S} within the target segment T may be determined by averaging the radiation intensities across the row(s) of pixels within the target segment T. In some embodiments, adjacent pixels of the first and second captured infrared images 402a, 402b may be grouped through pooling techniques, for example, during the reconciliation procedure. The generated radiation intensity profile 408 corresponds to a profile of emitted radiation from the glass wall W of the sample glass container C_{S} within the target segment T that extends around the glass container C_{S}. An example of the radiation intensity profile 408 is illustrated in FIG. 7, which shows the radiation intensity emitted from the glass wall W of the sample glass container C_{S} within a target segment located within the body portion 34 of the glass wall W of the container C_{S}. The radiation intensity profile 408 shown here is represented in a polar plot that depicts the radiation intensity of the glass container C_{S} within the target segment T using polar coordinates (r, θ) relative to the central longitudinal axis X of the container C_{S} as such a representation is easier to visualize in relation to the geometry of the actual container C_{S}. Of course, in other embodiments, the radiation intensity profile 408 could be represented in a Cartesian plot system using Cartesian coordinates (x, y).

The reconciliation procedure just described provides one avenue for allocating the radiation intensities observed in the 2D captured infrared images 402a, 402b to all of the 2n positions around the glass wall of the sample glass container C_{S}. The reconciliation procedure makes certain assumptions regarding the superposition of the front and back portions of the glass wall W in order to provide a systematic and simplified procedure for generating a numerical model. For example, in the reconciliation procedure, the effect of the curvature of the glass wall W when departing from the central longitudinal axis X of the glass wall W in the images 402a, 402b to the right and left boundaries of the images is not considered, nor is the impact of the slight angle change in horizontal angle when moving up and down the glass wall along the central longitudinal axis X. These variables are, in a sense, assumed to have no influence on the observed radiation intensity. The assumptions made in the reconciliation procedure can be made without loss of generality on the concept and as a first approximation. While the reconciliation procedure described herein is sufficient, other more sophisticated fits can be applied using, for example, machine learning or deep learning techniques to learn the A₁ and/or A₂ matrices from data.

After being imaged by the infrared image sensors 12a, 12b in the inspection zone 32, the sample glass container C_{S} is conveyed to the annealing lehr 28 on the forming conveyor 26 and is annealed in the annealing lehr 28. The sample glass container C_{S} is then removed from the forming conveyor 26 after the container C_{S} exits the annealing lehr 28 and brought to the offline measurement tool 42 in any suitable fashion. Alternately, the sample glass container C_{S} may be removed from the forming conveyor 26 prior to the container C_{S} entering the annealing lehr 28 and may be annealed individually such as, for example, in an oven. After annealing, the sample glass container C_{S} is analyzed by the offline measurement tool 42 and the thickness of the glass wall W is measured at least within the target segment T to obtain the calibrating thickness measurement(s) of the container C_{S} (step 350). One way in which the step of obtaining the calibrating thickness measurement(s) of the sample glass container C_{S} may be performed is shown, for example, in FIG. 4. Of course, other approaches for obtaining the calibrating thickness measurement(s) are possible, including approaches in which the offline thickness measurement tool 42 requires manual measurements to be taken.

The offline thickness measurement tool 42 measures the thickness of the glass wall W of the sample glass container C_{S} at one or more locations-preferably, at a set of various locations disposed around the central longitudinal axis X of the container C_{S}-within the target segment T of the container C_{S}. These measurements are the calibrating thickness measurement(s) 410. For example, as shown in FIG. 4, a set of calibrating thickness measurements 410 acquired from the offline thickness measurement tool 42 may be compiled in a data array. The data array may be a two-dimensional N×M array of numerical glass wall calibrating thickness measurements (reported here in inches) in which M is the number of different heights (a vertical position on the central longitudinal axis X) within the target segment T at which thickness measurements are taken and N is the number of thickness measurements taken at a given height. Here, in FIG. 4, the measurement data array is a 14×4 array of the calibrating thickness measurements 410, indicating that the thickness of the glass wall W is measured at fourteen different locations at each of the four different heights, although more or less thickness measurements may be obtained for purposes of step 350. The calibrating thickness measurement 410 of the glass wall W at each location can be determined, for example, through an automated analysis of the digital model of the sample glass container C_{S} that is executed by the offline thickness measurement tool 42, or by some other suitable approach depending on the design and capabilities of the tool 42. The calibrating thickness measurement(s) 410 are taken within the target segment T of the sample glass container C_{S} where the radiation intensity profile 408 is determined so that the radiation intensity profile 408 can be compared against and calibrated with relevant thickness data.

The radiation thickness correlation model 412 is determined (step 360) from the radiation intensity profile 408 and the calibrating thickness measurement(s) 410 of the sample glass container C_{S}, as shown in FIG. 4. The radiation thickness correlation model 412 may be determined by comparing the radiation intensity profile 408 of the sample glass container C_{S} against the calibrating thickness measurement(s) 410, ascertaining a correlation between the radiation intensity profile 408 and the calibrating thickness measurement(s) 410, and devising the radiation thickness correlation model 412 to represent or express the identified correlation. A variety of techniques may be employed to ascertain correlatives between the radiation intensity profile 408 and the calibrating thickness measurement(s) 410 such as functional data analysis and machine learning (ML) programs including, for instance, deep learning (DL) programs. If an ML program is employed, the radiation intensity profile 408 and the calibrating thickness measurement(s) 410 provided to the ML program as inputs are compared by the ML program, and the radiation thickness correlation model 412, which may include trainable parameters, is generated by the ML program and provided as an output. The trainable parameters of the radiation thickness correlation model 412 reflect the correlation between the inputs that the ML program has recognized and ascertained after comparing the inputs. As explained below, the radiation thickness correlation model 412 is processed as input in the thickness reading determination process 800 and enables an accurate translation of the radiation intensity profile of an examined glass container C_{E} into the thickness reading for that particular container C_{E} and, additionally, may be calibrated and refined whenever desired by repeating the radiation thickness correlation determination process 300.

The radiation thickness correlation model 412 is stored in computer memory, such as the memory 22 of the processing system 14, and enables the radiation intensity profile of each of the examined glass containers C_{E} to be translated into its corresponding thickness reading by the processing system 14 during the thickness reading determination process 800 (FIG. 8). If, for example, the radiation thickness correlation model 412 is determined by an ML program, the now-trained trainable parameters of the radiation thickness correlation model 412, after having been trained in the radiation thickness correlation determination process 300, are used to generate the thickness reading of the examined glass containers C_{E}. The thickness reading determination process 800 is performed for each glass container C_{E} being conveyed on the forming conveyor 26 that is designated for evaluation to determine the thickness reading of that particular container C_{E}. This may involve evaluating every glass container C on the forming conveyor 26 that passes through the evaluation zone 32 over a given period of time, or it may involve evaluating a defined subset of the glass containers C (e.g., every other glass container, every fifth glass container, a set of ten consecutive glass containers for every 100 containers, a set of 50 glass containers every hour, etc.) that pass through the evaluation zone 32 on the forming conveyor 26. Accordingly, while the thickness reading determination process 800 of FIG. 8 is discussed below in the context of a single examined glass container C_{E}, it is understood that the same process applies to each glass container C_{E} that is inspected.

In the thickness reading determination process 800, at least one infrared image 802a, 802b of the examined glass container C_{E} is captured by each of the infrared image sensors 12a, 12b (step 810) and the radiation intensity profile 808 of the examined glass container C_{E} is generated by the processing system 14 (step 820). The process for determining the radiation intensity profile 808 of the glass container C_{E} being examined during the thickness reading determination process 800 is preferably the same as the process for determining the radiation intensity profile 408 of the sample glass container C_{S} during the radiation thickness correlation determination process 300, 400 described above. Indeed, the steps of capturing the infrared images 802a, 802b of the examined glass container C_{E} (step 810) and generating the radiation intensity profile 808 of the examined glass container C_{E} (step 820) may be performed, and are preferably performed, in the same way that the corresponding steps (steps 310 and 320) of the radiation thickness correlation determination process 300 are performed as described above in connection with FIGS. 3-7. That discussion above, while not presently repeated in its entirety, is thus intended to apply to each examined glass container C_{E} as well and is therefore incorporated into this discussion of the thickness reading determination process 800 and to FIG. 8 as if fully restated here. After the examined glass container C_{E} has been imaged by the infrared image sensors 12a, 12b and its radiation intensity profile 808 has been generated, the thickness reading of the examined glass container C_{E} is determined (step 830).

The thickness reading of the examined glass container C_{E} is determined in step 830 based on the radiation intensity profile 808 of the container C_{E} and the radiation thickness correlation model 412. As discussed above, the radiation thickness correlation model 412 is stored in the memory 22 of the processing system 14 as any appropriate data structure and is accessed (recalled from the memory 22 for processing) by the processing system 14 when calculating the thickness reading of the examined glass container C_{E}. The radiation thickness correlation model 412 converts the radiation intensity profile 808 of the examined glass container C_{E} generated during step 820 into the thickness reading of the container C_{E}. The thickness profile presented in the thickness reading is representative of the predicted thickness of the glass wall W of the examined glass container C_{E} around the central longitudinal axis X of the container C_{E} within the target segment T. For any location of the glass wall W within the predicted thickness profile, the thickness profile provides an inferred numerical thickness value of the glass wall W of the examined glass container C_{E} at that location. The thickness reading of the examined glass container C_{E} may be stored in a production database along with a container identifier (CID) of the examined glass container C_{E}, such as a production plant and serial number, for example, if desired.

The thickness reading determination process 800 may be performed continuously or periodically for glass containers C that are being conveyed on the forming conveyor 26 in the hot end 16 of the glass container manufacturing system 18. The execution of the thickness reading determination process 800 allows the hot end glass container evaluation system 10 to provide real-time, accurate, remote, and non-contact glass wall distribution data for the glass containers C_{E} being examined without disrupting the manufacturing process in the hot end 16. The obtained thickness readings, in turn, can be analyzed, either manually or by a computer program, to timely uncover problematic trends in the glass distribution of the glass containers C, out-of-specification thickness defects, or other potential issues that would cause an unacceptable quantity of the manufactured glass container(s) C to fail to meet expected manufacturing specifications. Based on this analysis, one or more process conditions in the hot end 16 may be adjusted to correct the underlying contributing cause(s) of the identified deviations. The radiation thickness correlation model 412 may be periodically calibrated by executing the radiation thickness correlation determination process 300 to help ensure that the thickness readings being output by the hot end glass container evaluation system 10 are accurate in view of the calibrating thickness measurement(s) obtained for the sample glass containers C_{S} that are measured by the offline thickness measurement tool 42.

As used in herein, the terminology “for example,” “e.g.,” for instance,” “like,” “such as,” “comprising,” “having,” “including,” and the like, when used with a listing of one or more elements, is to be construed as open-ended, meaning that the listing does not exclude additional elements. Also, as used herein, the term “may” is an expedient merely to indicate optionality, for instance, of a disclosed embodiment, element, feature, or the like, and should not be construed as rendering indefinite any disclosure herein. Finally, the subject matter of this application is presently disclosed in conjunction with several explicit illustrative embodiments and modifications to those embodiments, using various terms. All terms used herein are intended to be merely descriptive, rather than necessarily limiting, and are to be interpreted and construed in accordance with their ordinary and customary meaning in the art, unless used in a context that requires a different interpretation. And for the sake of expedience, each explicit illustrative embodiment and modification is hereby incorporated by reference into one or more of the other explicit illustrative embodiments and modifications. The present disclosure is intended to embrace all such embodiments and modifications of the subject matter of this application, and equivalents thereto, as fall within the broad scope of the accompanying claims.

1. A method of evaluating a glass container in a hot end of a glass container manufacturing system, the method comprising:
obtaining one or more calibrating thickness measurements of at least one sample glass container;
generating a radiation intensity profile of the sample glass container;
determining a radiation thickness correlation model based on the one or more calibrating thickness measurements of the sample glass container and the radiation intensity profile of the sample glass container;
generating a radiation intensity profile for an examined glass container while the examined glass container is in a hot end of a glass container manufacturing system; and
determining a thickness reading of the examined glass container based on the radiation intensity profile of the examined glass container and the radiation thickness correlation model.

2. The method set forth in claim 1, wherein the one or more calibrating thickness measurements is a set of calibrating thickness measurements from various locations disposed around a central longitudinal axis of the sample container.

3. The method set forth in claim 1, wherein the thickness reading of the examined glass container is a thickness profile of a glass wall of the examined glass container around a central longitudinal axis of the examined glass container.

4. The method set forth in claim 1, wherein each of the sample glass container and the examined glass container has a glass wall that includes a cylindrical body portion.

5. The method set forth in claim 1, wherein the one or more calibrating thickness measurements of the sample glass container and the radiation intensity profile of the sample glass container are obtained within a target segment of the sample glass container, and wherein the radiation intensity profile of the examined glass container is generated within a corresponding target segment of the examined glass container.

6. The method set forth in claim 1, wherein generating the radiation intensity profile of the sample glass container comprises capturing at least two angularly displaced infrared images of the sample glass container in the hot end of the glass container manufacturing system and generating the radiation intensity profile of the sample glass container from the infrared images of the sample glass container, and wherein generating the radiation intensity profile of the examined glass container comprises capturing at least two angularly displaced infrared images of the examined glass container in the hot end of the glass container manufacturing system and generating the radiation intensity profile of the examined glass container from the infrared images of the examined glass container.

7. The method set forth in claim 1, wherein determining the radiation thickness correlation model comprises inputting the radiation intensity profile of the sample glass container and the one or more calibrating thickness measurements of the sample glass container into a machine learning program that ascertains a correlation between the radiation intensity profile of the sample glass container and the one or more calibrating thickness measurements of the sample glass container to generate the radiation thickness correlation model.

8. The method set forth in claim 1, wherein obtaining the one or more calibrating thickness measurements comprises acquiring the one or more calibrating thickness measurements with an offline thickness measurement tool.

9. The method set forth in claim 8, wherein the offline thickness measurement tool includes a computed tomography scanner.

10. A method of evaluating a glass container in a hot end of a glass container manufacturing system, the method comprising:
producing glass containers in a hot end of a glass container manufacturing system;
generating a radiation intensity profile of an examined glass container, the radiation intensity profile of the examined glass container being located within a target segment of the examined glass container;
determining a thickness reading of the examined glass container, the thickness reading of the examined glass container being based on the radiation intensity profile of the examined glass container and a radiation thickness correlation model that translates the radiation intensity profile of the examined glass container into the thickness reading of the examined glass container.

11. The method set forth in claim 10, wherein generating the radiation intensity profile comprises capturing at least a first infrared image and a second infrared image of the examined glass container in the hot end of the glass container manufacturing system, the first infrared image presenting a first portion of the examined glass container and the second infrared image presenting a second portion of the examined glass container that is angularly displaced from the first portion of the examined glass container about a central longitudinal axis of the examined glass container.

12. The method set forth in claim 11, comprising:
capturing the first infrared image with a first infrared image sensor; and
capturing the second infrared image with a second infrared image sensor that is angularly offset from the first infrared image sensor.

13. The method set forth in claim 10, wherein the radiation thickness correlation model is determined by a radiation thickness correlation determination process that comprises:
generating a radiation intensity profile of a sample glass container, the radiation intensity profile of the sample glass container being located within a target segment of the sample glass container that corresponds to the target segment of the examined glass container;
obtaining one or more calibrating thickness measurements of the sample glass container within the target segment of the sample glass container;
comparing the radiation intensity profile of the sample glass container and the one or more calibrating thickness measurements of the sample glass container to ascertain a correlation between the radiation intensity profile of the sample glass container and the one or more calibrating thickness measurements of the sample glass container; and
generating the radiation thickness correlation model based off of the correlation between the radiation intensity profile of the sample glass container and the one or more calibrating thickness measurements of the sample glass container.

14. The method set forth in claim 13, wherein comparing the radiation intensity profile of the sample glass container and the one or more calibrating thickness measurements of the sample glass container comprises inputting the radiation intensity profile of the sample glass container and the one or more calibrating thickness measurements of the sample glass container into a machine learning program that ascertains the correlation between the radiation intensity profile of the sample glass container and the one or more calibrating thickness measurements of the sample glass container, and wherein generating the radiation thickness correlation model comprises outputting the radiation thickness correlation model from the machine learning program with the radiation thickness correlation model reflecting the correlation between the radiation intensity profile of the sample glass container and the one or more calibrating thickness measurements of the sample glass container.

15. The method set forth in claim 13, wherein obtaining one or more calibrating thickness measurements of the sample glass container comprises:
bringing the sample glass container to an offline thickness measurement tool; and
acquiring the one or more calibrating thickness measurements with the offline thickness measurement tool.

16. The method set forth in claim 15, wherein the offline thickness measurement tool includes a computed tomography scanner.

17. The method set forth in claim 13, wherein the one or more calibrating thickness measurements is a set of calibrating thickness measurements taken from a plurality locations of the sample glass container within the target segment of the sample glass container.

18. The method set forth in claim 10, wherein the thickness reading of the examined glass container is a thickness profile of a glass wall of the examined glass container around a central longitudinal axis of the examined glass container within the target segment.

19. A method of operating a hot end glass container evaluation system, the method comprising:
(a) generating a radiation intensity profile of at least one sample glass container within a target segment of the sample glass container;
(b) obtaining one or more calibrating thickness measurements of the at least one sample glass container within the target segment of the sample glass container;
(c) determining a radiation thickness correlation model based on the one or more calibrating thickness measurements of the at least one sample glass container and the radiation intensity profile of the at least one sample glass container;
(d) generating a radiation intensity profile for at least one examined glass container within a target segment of the examined glass container that corresponds to the target segment of the sample glass container;
(e) determining a thickness reading of the at least one examined glass container based on the radiation intensity profile of the examined glass container and the radiation thickness correlation model; and
(f) calibrating the radiation thickness correlation model by repeating steps (a)-(c) for at least one other sample glass container.

20. The method set forth in claim 19, wherein generating the radiation intensity profile of the at least one sample glass container in step (a) comprises capturing at least two angularly displaced infrared images of the at least one sample glass container in a hot end of a glass container manufacturing system and generating the radiation intensity profile of the at least one sample glass container from the infrared images of the at least one sample glass container, and wherein generating the radiation intensity profile of the at least one examined glass container in step (d) comprises capturing at least two angularly displaced infrared images of the at least one examined glass container in the hot end of a glass container manufacturing system and generating the radiation intensity profile of the at least one examined glass container from the infrared images of the at least one examined glass container.

21. The method set forth in claim 19, wherein determining the radiation thickness correlation model in step (c) comprises:
comparing the radiation intensity profile of the at least one sample glass container and the one or more calibrating thickness measurements of the at least one sample glass container to ascertain a correlation between the radiation intensity profile of the at least one sample glass container and the one or more calibrating thickness measurements of the at least one sample glass container; and
generating the radiation thickness correlation model based off of the correlation between the radiation intensity profile of the at least one sample glass container and the one or more calibrating thickness measurements of the at least one sample glass container.

22. The method set forth in claim 21, wherein comparing the radiation intensity profile of the at least one sample glass container and the one or more calibrating thickness measurements of the at least one sample glass container comprises inputting the radiation intensity profile of the at least one sample glass container and the one or more calibrating thickness measurements of the at least one sample glass container into a machine learning program that compares the radiation intensity profile of the at least one sample glass container and the one or more calibrating thickness measurements of the at least one sample glass container and ascertains the correlation between the radiation intensity profile of the at least one sample glass container and the one or more calibrating thickness measurements of the at least one sample glass container.

23. The method set forth in claim 19, wherein obtaining the one or more calibrating thickness measurements in step (b) comprises acquiring the one or more calibrating thickness measurements of the at least one sample glass container with an offline thickness measurement tool.

24. A hot end glass container evaluation system, comprising:
at least two infrared image sensors each being configured to capture an infrared image of an examined glass container in a hot end of a glass container manufacturing system; and
a processing system configured to receive the captured infrared images of the examined glass container from the infrared image sensors and further comprising at least one processor and memory storing computer instructions that, when executed by the at least one processor, cause the processing system to:
generate a radiation intensity profile of the examined glass container from the infrared images captured by the infrared image sensors; and
determine a thickness reading of the examined glass container based on the radiation intensity profile of the examined glass container and a radiation thickness correlation model, stored in the memory, that translates the radiation intensity profile of the examined glass container into the thickness reading of the examined glass container.

25. The hot end glass container evaluation system set forth in claim 24, wherein the at least two infrared image sensors comprises a first infrared image sensor configured to capture a first infrared image of a first portion of the examined glass container and a second infrared image sensor configured to capture a second infrared image of a second portion of the examined glass container that is angularly displaced from the first portion of the examined glass container.

26. The hot end glass container evaluation system set forth in claim 24, further comprising an offline thickness measurement tool.

27. The hot end glass container evaluation system set forth in claim 26, wherein the radiation thickness correlation model is representative of a correlation between a radiation intensity profile of at least one sample glass container and one or more calibrating thickness measurements of the at least one sample glass container acquired from the offline measurement tool.
